(19) United States
(12) Patent Application Publication
(10) Pub. No.: US 2022/0015865 A1
(43) Pub. Date: Jan. 20, 2022

(54) GUIDED IMPLANT DRILL SYSTEM AND METHODS OF USE

(71) Applicant: DANIEL S. KIM, VANCOUVER, WA (US)

(72) Inventor: DANIEL S. KIM, VANCOUVER, WA (US)

(21) Appl. No.: 17/377,133

(22) Filed: Jul. 15, 2021

Related U.S. Application Data
(60) Provisional application No. 63/052,067, filed on Jul. 15, 2020.

(51) Int. Cl. A61C 1/08

(52) U.S. Cl. CPC A61C 1/084

(57) ABSTRACT

A guided implant drill system includes a guided implant drill bit with a cutting cylinder having a cutting diameter corresponding to a selected dental implant anchor and a noncutting guide portion having a diameter corresponding to a selected pilot drill bit, and a corresponding guided implant final drill bit having a noncutting guide cylinder portion and a cutting tip extending therefrom, with final drill bit noncutting cylinder portion and cutting tip diameters matching the guided implant drill bit cutting cylinder, and the final drill bit cutting tip depth matching the guided implant drill bit guide portion depth. A method of use for the guided implant drill system is also provided.

### CROSS-REFERENCE TO RELATED APPLICATIONS

This application is a nonprovisional of, and claims priority to, co-pending application 63/052,067 filed Jul. 15, 2020, all of which is hereby incorporated by reference into this application.

### FIELD OF THE INVENTION

The present invention relates to dental surgical tools and methods. More particularly, the present invention relates to a novel design for a system of drilling tools for performing dental implant surgeries, and methods for their use.

### BACKGROUND OF THE INVENTION

Conventional apparatus and methods for performing dental implant surgery are very basic, and vulnerable to errors created by the essentially free-hand drilling apparatus and methods widely used. The conventional procedure, after resecting the gum tissue, is to drill a pilot hole into the alveolar bone using a conventional bladed dental drill bit of small diameter, and then to drill successively wider holes using progressively larger conventional bladed drill bits until the required diameter to receive the intended implant anchor is achieved. Exemplary embodiments of a conventional pilot drill bit and prior art 5 mm implant drill bit are shown in FIGS. 1-2. As can be seen, there is nothing in their structure which would assist the surgeon in maintaining consistent alignment or hole diameter from step-to-step, especially if the surgeon attempted to switch immediately to using a large-diameter drill bit (e.g. in the 4 mm-8 mm range) after providing only a 2 mm or 2.2 mm pilot hole because the small-diameter pilot hole would provide little-to-no directional stability. The unguided free-hand nature of conventional dental drill bits requires multiple, incrementally increasing diameter, free-hand drilling steps.

Although the initial pilot hole provides some assistance in guiding the surgeon, each step essentially amounts to another free-handed drilling operation which introduces additional likelihood of errors in the angular and lateral displacement of the implant hole, with negative consequences for the patient—sometimes potentially severe consequences. Each drilling step introduces additional risk of causing unintended damage to the alveolar bone, through overheating and/or fracturing. Additionally, the requirement for multiple drilling steps substantially increases the time required to perform the procedure, thereby increasing the time the patient is subject to anesthesia—which increases surgical risk and patient discomfort—and reducing the number of patients the surgeon is able to assist in a given block of time, which increases costs.

There is a need for an improved dental surgical drill system which reduces risk of misalignment errors, reduces the stress imposed on the alveolar bone during drilling procedures, and reduces the time required to perform such procedures.

### SUMMARY AND ADVANTAGES

A guided implant drill system includes a bit having a central axis, a shank portion, a cutting cylinder portion and a guide portion having a guiding tip diameter matching a selected guide drill bit, each aligned along the central axis; the shank portion adapted to couple to a dental drill driver, the shank extending from a shank first end to a shank second end; the cutting cylinder portion extending from a cutting cylinder portion first end rigidly connected to the shank portion second end to a cutting cylinder portion second end along the central axis, the cutting cylinder portion including a plurality of flutes defining a plurality of cutting edges, the cutting edges defining the full diameter of the cutting cylinder portion and selected to correspond to a selected dental implant anchor; the guide portion extending from the cutting cylinder portion second end and including a constant diameter extending along its axial length, the guide portion diameter corresponding to a selected pilot drill bit cutting diameter, the guide portion including a smooth noncutting surface, the guide portion further including at least one vent groove extending axially along its surface and a rounded tip, the vent groove in fluid communication with at least one cutting cylinder trench.

The guided implant drill system may include the guide cylinder portion extending from a guide cylinder first end, connected to the shank portion second end, to a guide cylinder second end connected to the cutting tip and including a plurality of noncutting helical guide cylinder portion flutes and corresponding guide cylinder portion trenches each extending from the guide cylinder portion first end to the guide cylinder portion second end, the guide cylinder portion flutes defining a guide cylinder portion diameter; the final drill cutting tip extending from a base connected to the guide cylinder portion second end to a terminal end and including a plurality of cutting flutes and corresponding cutting tip trenches extending from the cutting tip base to the cutting tip terminal end, each of the plurality of cutting tip trenches in fluid communication with a corresponding guide cylinder portion trench.

A method for performing an osteotomy in preparation for installing a dental implant anchor using a set of a guided implant drill bit and guided implant final drill bit includes the steps of:

a. providing a guided implant drill system as in Claim 3 corresponding to the selected dental implant anchor size;

b. creating a pilot hole at the implant location using the selected pilot drill bit, until a predetermined depth is achieved thereby creating an osteotomy terminal depth at the osteotomy site;

c. using the first set guided implant drill bit coupled to a dental driver, inserting the first set guided implant drill bit guide portion into the pilot hole and drilling into the bone until the guided implant drill bit guide portion terminal end reaches the osteotomy terminal depth, thereby creating an osteotomy ridge; and,

d. using the first set guided implant final drill bit, inserting the first set guided implant final drill bit into the osteotomy site against the osteotomy ridge and drilling until the guided implant final drill bit cutting tip terminal end reaches the osteotomy terminal depth, thereby removing the osteotomy ridge.

The guided implant drill system described and claimed provides important advantages over existing apparatus and methods, including but not limited to: (1) providing directional support for a dental surgeon in carrying out the widening portion of an osteotomy in preparation for a dental implant while providing channels for irrigation to minimize risk of bone necrosis due to heat generation and provide efficient debris removal; (2) reducing the number of drilling cycles required to complete a preparatory osteotomy; and, (3) providing apparatus and means to finish the lower portion of a preparatory osteotomy with minimum risk of unintentional additional removal of bone from the upper portion of the osteotomy location, while providing channels for irrigation to minimize risk of bone necrosis and provide efficient debris removal.

### BRIEF DESCRIPTION OF THE DRAWINGS

The accompanying drawings, which are incorporated into and constitute a part of this specification, illustrate one or more embodiments of the invention and, together with the detailed description, serve to explain the principles and implementations of the invention.

FIG. 1 is a view of a prior art dental implant drill bit of 5 mm diameter.

FIG. 2 is a side view of a conventional dental pilot drill bit P.

FIG. 2A is an end-on view of a conventional pilot drill bit P.

FIG. 3 shows a side view of a first embodiment of a guided implant drill bit.

FIG. 4 shows a side view of a first embodiment of a guided implant drill bit.

FIG. 5 shows a sideview of a first embodiment of a guided implant drill bit.

FIG. 5A shows an end view of a first embodiment of a guided implant drill bit.

FIG. 6 shows a sideview of a first embodiment of a guided implant finishing drill bit.

FIG. 7 shows a sideview of a first embodiment of a guided implant finishing drill bit.

FIG. 8 shows a sideview of a second embodiment of a guided implant drill bit of 3 mm cutting diameter.

FIG. 9 shows a sideview of a second embodiment of a guided implant final drill bit of 3 mm cutting diameter.

FIG. 10 shows a sideview of a third embodiment of a guided implant drill bit of 4 mm cutting diameter.

FIG. 11 shows a sideview of a third embodiment of a guided implant final drill bit of 4 mm cutting diameter.

FIG. 12 shows a simplified view of an osteotomy procedure, with the pilot drill bit P preparing to engage the alveolar bone.

FIG. 13 shows a simplified cutaway view of an osteotomy procedure, with the pilot drill bit P a its terminal depth T.

FIG. 14 shows a simplified cutaway view of an osteotomy procedure, with the guided implant drill bit 100 preparing to engage the alveolar bone through the pilot hole.

FIG. 15 shows a simplified cutaway view of an osteotomy procedure, with the guided implant drill bit 100 penetrated to terminal point T with osteotomy final diameter of 5 mm.

FIG. 16 shows a simplified cutaway view of an osteotomy procedure, with the guided implant final drill bit 200 preparing to engage the alveolar bone through the osteotomy final penetration.

FIG. 17 shows a simplified cutaway view of an osteotomy procedure, with the guided implant final drill bit 200 approaching osteotomy ridge R.

FIG. 18 shows a simplified cutaway view of an osteotomy procedure, with the guided implant final drill bit 200 penetrated to osteotomy terminal depth T.

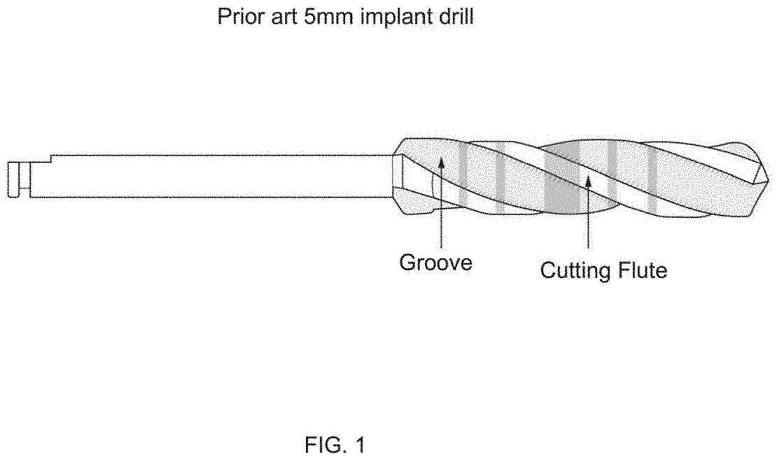

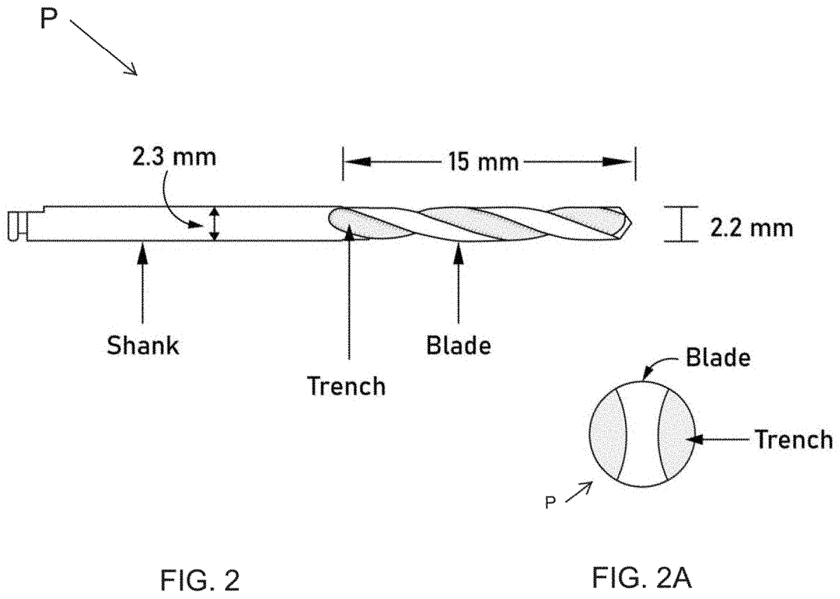

### REFERENCE NUMBERS

The following table provides a summary of the Reference Numbers used in the Detailed Description and the Drawings:

| Ref. Nr. | Description |
|---|---|
| 10 | First Embodiment |
| 100 | Guided Implant Drill Bit |
| 102 | Shank Portion |
| 104 | Cutting Cylinder Portion |
| 106 | Guide Portion |
| 108 | Rotational Axis |
| 110 | Shank Portion First End |
| 112 | Shank Portion Second End |
| 114 | Cutting Cylinder Portion First End |
| 116 | Cutting Cylinder Portion Second End |
| 118 | Cutting Cylinder First Helical Flute |
| 120 | Cutting Cylinder Second Helical Flute |
| 122 | First Flute First End |
| 124 | Second Flute First End |
| 126 | First Flute Second End |
| 128 | Second Flute Second End |
| 130 | Cutting Cylinder First Helical Trench |
| 132 | Cutting Cylinder Second Helical Trench |
| 134 | First Trench First End |
| 136 | Second Trench First End |
| 138 | First Trench Second End |
| 140 | Second Trench Second End |
| 142 | First Flute Terminal Outer Edge |
| 144 | Second Flute Terminal Outer Edge |
| 146 | Guide Portion First End |
| 148 | Guide Portion Terminal End |
| 150 | Guide Portion Vent Groove |
| 152 | Vent Groove First End |
| 154 | Vent Groove Second End |
| 156 | Guide Portion Terminal End Ogive Base |
| 158 | Guide Portion Diameter |
| 160 | Cutting Cylinder Cutting Diameter |
| 162 | Cutting Cylinder Depth Markings |
| 164 | Guide Portion Outer Surface |
| 166 | Guide Portion Length |
| 168 | Cutting Cylinder Length |
| 200 | Guided Implant Final Drill Bit |
| 202 | Shank Portion |
| 204 | Noncutting Guide Cylinder Portion |
| 206 | Cutting Tip |
| 208 | Rotational Axis |
| 210 | Shank Portion First End |
| 212 | Shank Portion Second End |
| 214 | Guide Cylinder Portion First End |
| 216 | Guide Cylinder Portion Second End |
| 218 | First Noncutting Helical Flute |
| 220 | Second Noncutting Helical Flute |
| 222 | First Noncutting Flute First End |
| 224 | Second Noncutting Flute First End |
| 226 | First Noncutting Flute Second End |
| 228 | Second Noncutting Flute Second End |
| 230 | First Helical Trench |
| 232 | Second Helical Trench |
| 234 | First Helical Trench First End |
| 236 | Second Helical Trench First End |
| 238 | First Helical Trench Second End |
| 240 | Second Helical Trench Second End |
| 242 | [Not used] |
| 244 | [Not used] |
| 246 | Cutting Tip First Portion |
| 248 | Cutting Tip Terminal End |
| 250 | [Not used] |
| 252 | [Not used] |
| 254 | [Not used] |
| 256 | [Not used] |
| 258 | Cutting Tip Diameter |
| 260 | Guide Cylinder Portion Diameter |
| 262 | [Not used] |
| 264 | [Not used] |
| 266 | [Not used] |
| 268 | First Noncutting Helical Flute Leading Edge |
| 270 | Second Noncutting Helical Flute Leading Edge |
| 272 | First Noncutting Helical Flute Trailing Edge |
| 274 | Second Noncutting Helical Flute Trailing Edge |
| 276 | Guide Cylinder Portion Length |
| 278 | Cutting Tip First Cutting Flute |
| 280 | Cutting Tip Second Cutting Flute |
| 282 | Cutting Tip First Trench |
| 284 | Cutting Tip Second Trench |
| 286 | Cutting Tip First Cutting Flute Leading Edge |
| 288 | Cutting Tip Second Cutting Flute Leading Edge |
| 290 | Cutting Tip First Cutting Flute Trailing Edge |
| 292 | Cutting Tip Second Cutting Flute Trailing Edge |
| 300 | Guided Implant Drill Bit Second Embodiment |
| 304 | Cutting Cylinder Portion |
| 306 | Guide Portion |
| 318 | Cutting Cylinder First Helical Cutting Flute |
| 320 | Cutting Cylinder Second Helical Cutting Flute |
| 330 | Cutting Cylinder First Helical Trench |
| 332 | Cutting Cylinder Second Helical Trench |
| 350 | Guide Portion Vent Groove |
| 400 | Guided Implant Final Drill Bit Second Embodiment |
| 404 | Guide Cylinder Portion |
| 406 | Cutting Tip |
| 418 | First Noncutting Helical Flute |
| 420 | Second Noncutting Helical Flute |
| 430 | First Helical Trench |
| 432 | Second Helical Trench |
| 500 | Guided Implant Drill Bit Third Embodiment |
| 504 | Cutting Cylinder Portion |
| 506 | Guide Portion |
| 518 | Cutting Cylinder First Helical Cutting Flute |
| 520 | Cutting Cylinder Second Helical Cutting Flute |
| 530 | Cutting Cylinder First Helical Trench |
| 532 | Cutting Cylinder Second Helical Trench |
| 550 | Guide Portion Vent Groove |
| 600 | Guided Implant Final Drill Bit Third Embodiment |
| 604 | Guide Cylinder Portion |
| 606 | Cutting Tip |
| 618 | First Noncutting Helical Flute |
| 620 | Second Noncutting Helical Flute |
| 630 | First Helical Trench |
| 632 | Second Helical Trench |

### DETAILED DESCRIPTION

Before beginning a detailed description of the subject invention, mention of the following is in order. When appropriate, like reference materials and characters are used to designate identical, corresponding, or similar components in different figures. The figures associated with this disclosure typically are not drawn with dimensional accuracy to scale, i.e., such drawings have been drafted with a focus on clarity of viewing and understanding rather than dimensional accuracy.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application and business related constraints, and that these specific goals will vary from one implementation to another and from one developer to another. Moreover, it will be appreciated that such a development effort might be complex and time-consuming but would nevertheless be a routine undertaking of engineering for those of ordinary skill in the art having the benefit of this disclosure.

Use of directional terms such as “upper,” “lower,” “above,” “below”, “in front of,” “behind,” etc. are intended to describe the positions and/or orientations of various components of the invention relative to one another as shown in the various Figures and are not intended to impose limitations on any position and/or orientation of any embodiment of the invention relative to any reference point external to the reference.

Those skilled in the art will recognize that numerous modifications and changes may be made to the exemplary embodiment(s) without departing from the scope of the claimed invention. It will, of course, be understood that modifications of the invention, in its various aspects, will be apparent to those skilled in the art, some being apparent only after study, others being matters of routine mechanical, chemical and electronic design. No single feature, function or property of the exemplary embodiment(s) is essential. Other embodiments are possible, their specific designs depending upon the particular application. As such, the scope of the invention should not be limited by the particular embodiments herein described but should be defined only by the appended claims and equivalents thereof.

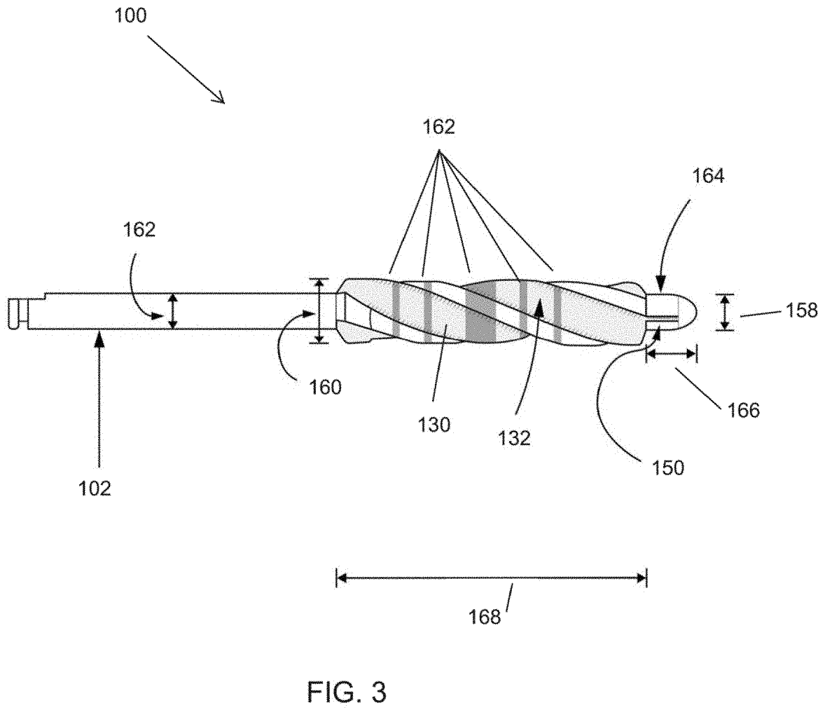

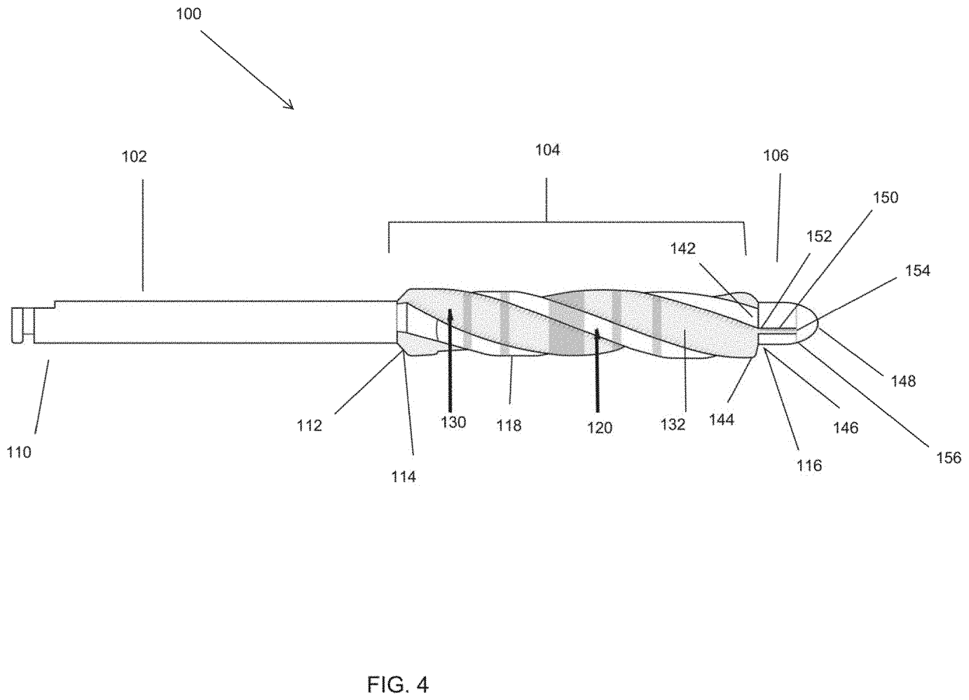

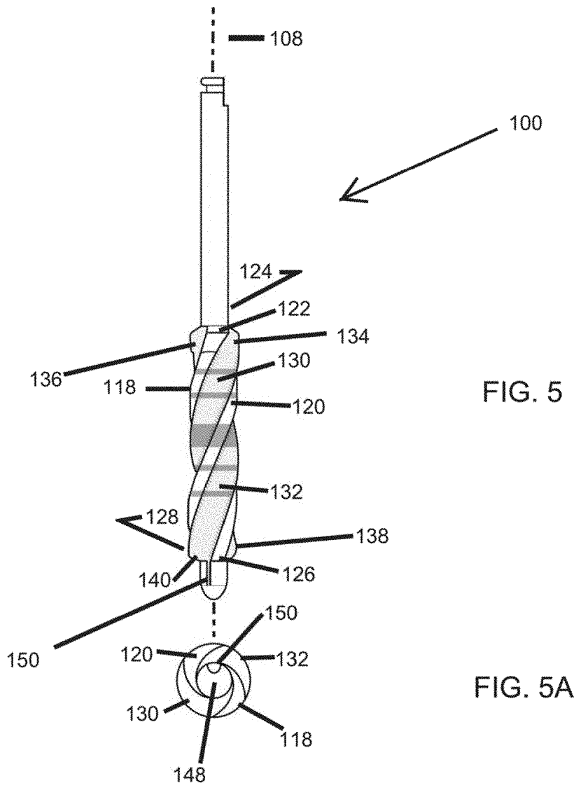

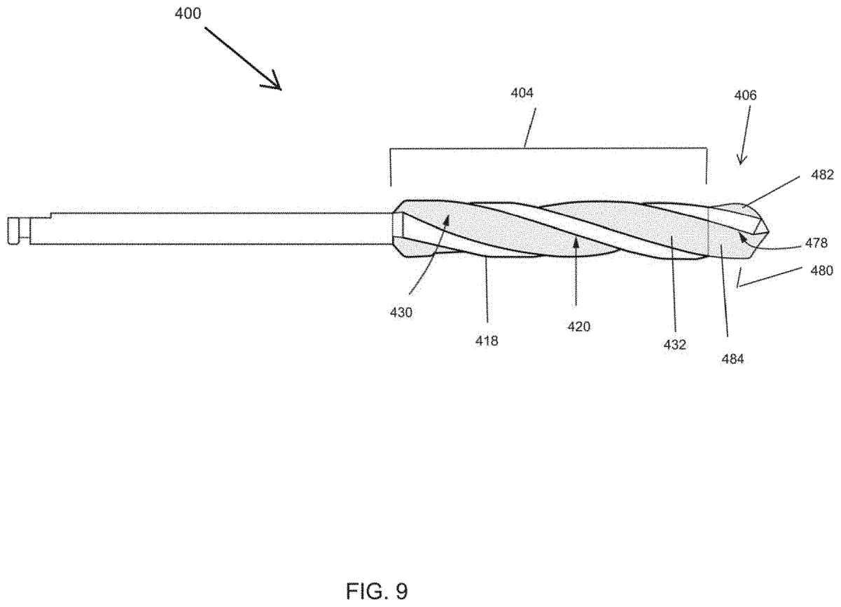

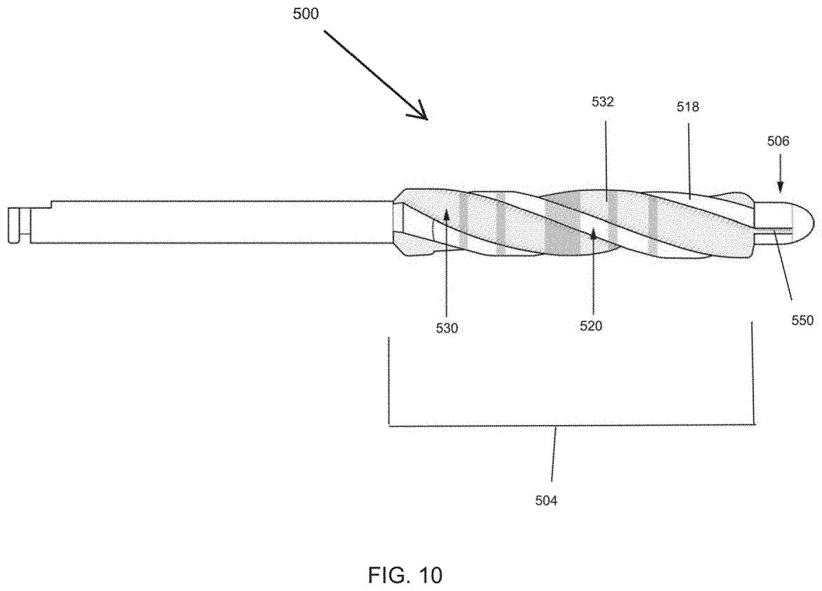

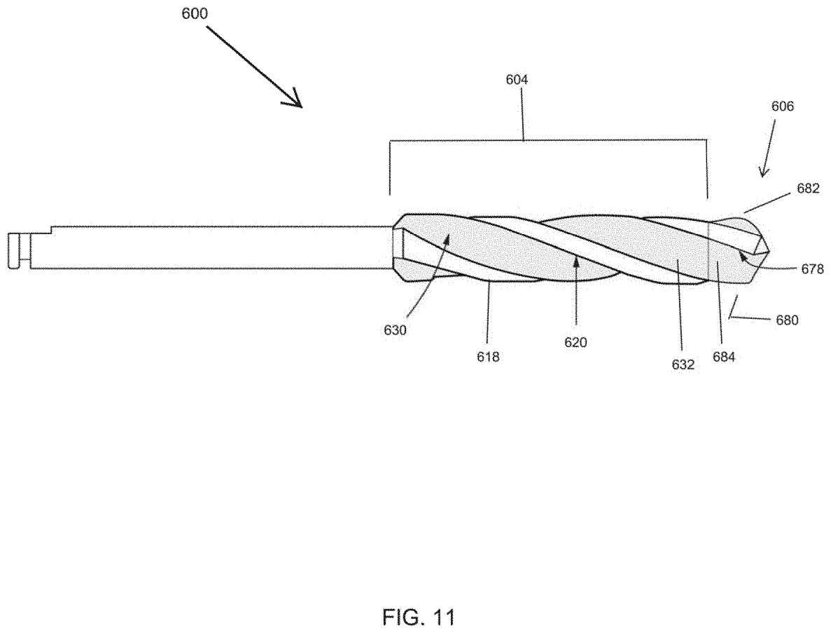

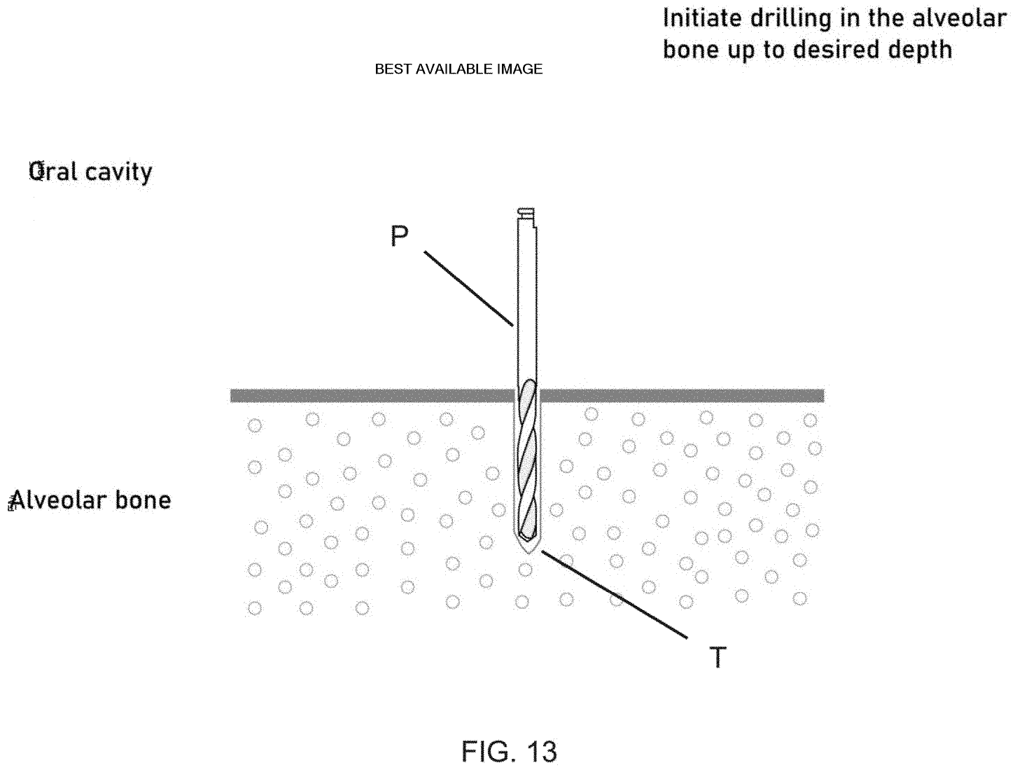

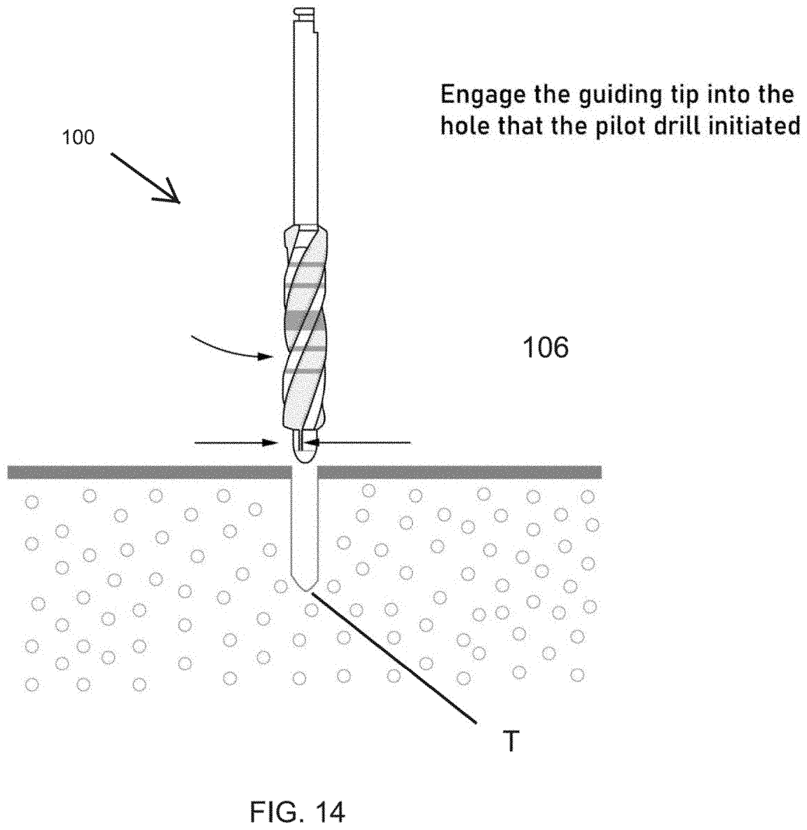

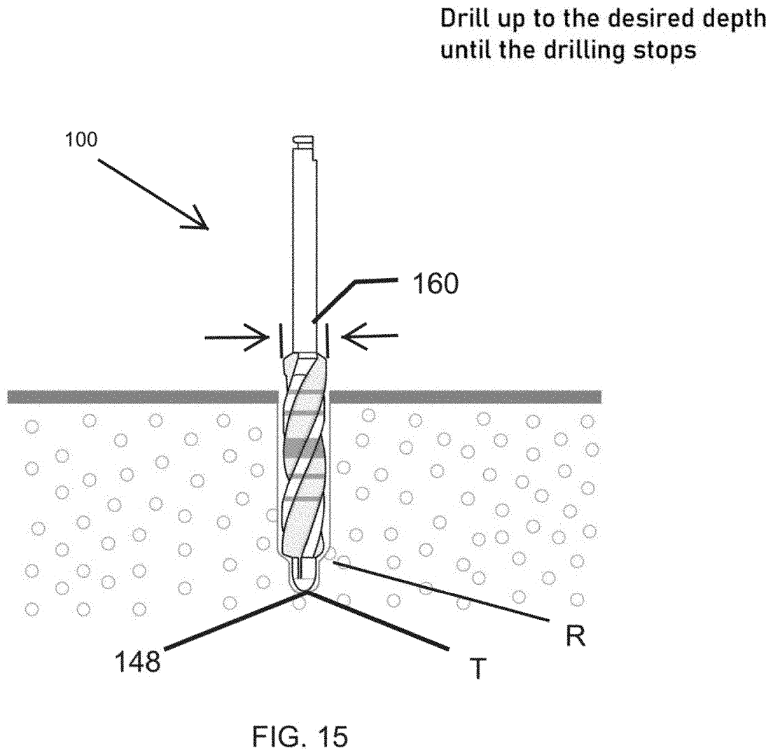

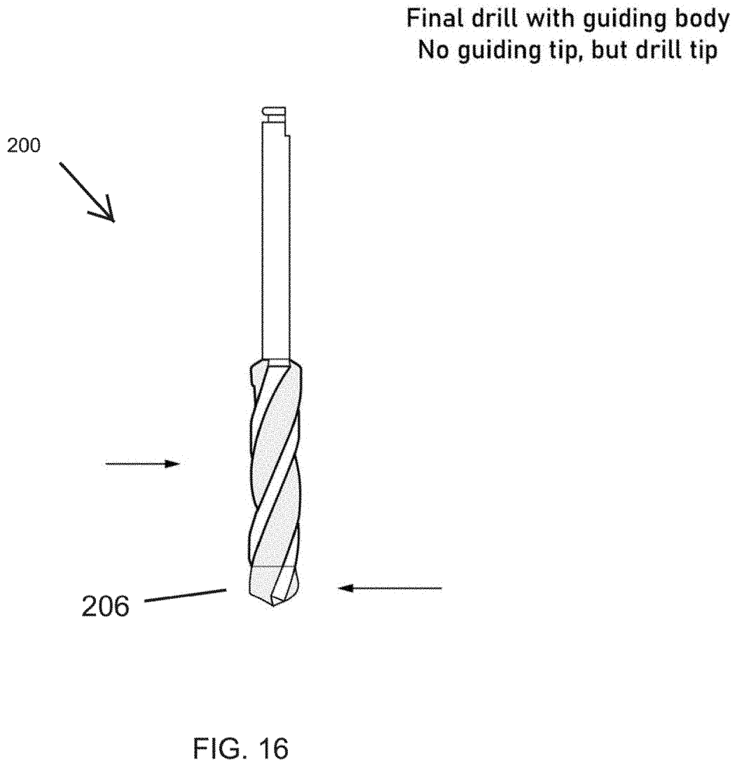

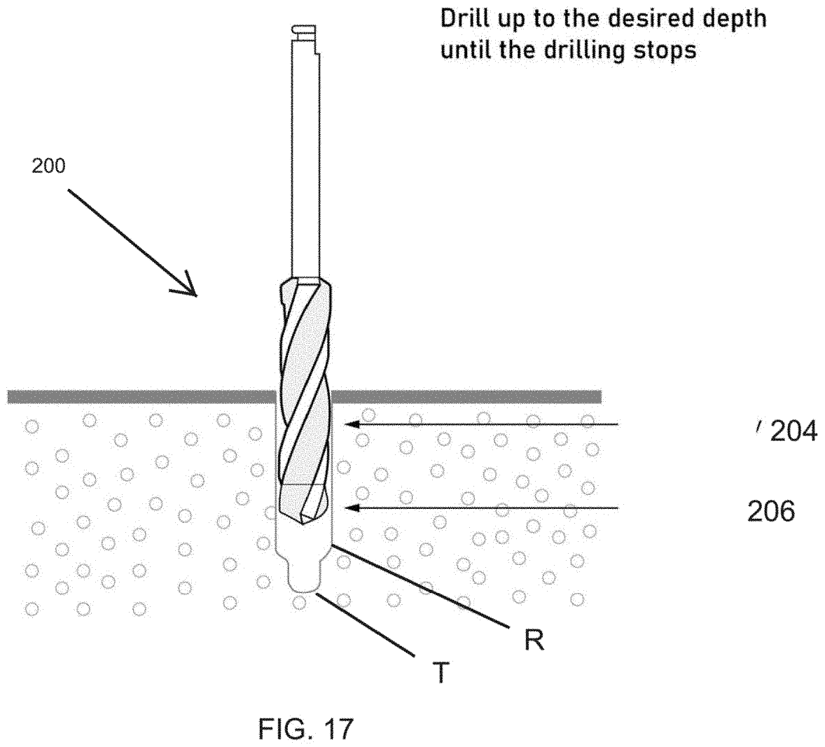

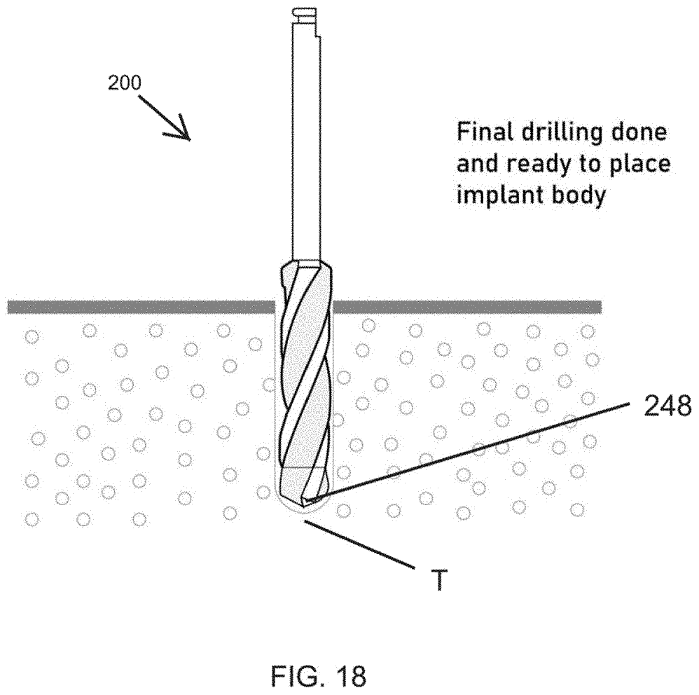

Referring to FIGS. 3-18, an embodiment of a guided implant drill system 10 and its method of use are shown. In the embodiment, the system includes at least one guided implant drill bit 100 and a final drill bit 200, which are used in conjunction with a selected pilot drill bit P to create a properly aligned and depth-controlled osteotomy into the alveolar bone to receive a dental implant anchor. The system may include sets of guided drill bits 100, 300, 500 with corresponding final drill bits 200, 400, 600 corresponding to dental implant anchors of different sizes, for use with a selected diameter pilot drill bit P. In the embodiment, pilot drill bit P has cutting diameter of 2.2 mm, which is typical in the field, but a different pilot drill diameter and corresponding guide tip diameter may be selected as well.

Referring to FIGS. 3-5A, a first embodiment of a guided implant drill bit 100 is shown, for use in conjunction with a pilot drill bit P of approximately 2.2 mm diameter. Guided implant drill bit 100 includes a shank portion 102, a cutting cylinder portion 104 and a guide portion 106. Shank portion 102 is adapted to couple to a selected dental drill driver or class of dental drivers, the shank portion extending from a shank first end 110 to a shank second end 112. The centerlines of shank portion 102, cutting cylinder portion 104 and guide portion 106 align and together define the rotational axis 108 of the guided implant drill bit 100.

Cutting cylinder portion 104 extends along rotational axis 108 from cutting cylinder portion first end 114 rigidly connected to shank portion second end 112 to cutting cylinder portion second end 116, defining a cutting cylinder length 168. Cutting cylinder portion 104 includes a plurality of helical flutes 118, 120, each extending from a flute first end 122, 124 (not visible in view), respectively, to a flute second end 126, 128, respectively. In the embodiment, flutes 118, 120 are disposed on opposed sides of cutting cylinder portion 104 at 180° intervals, defining an equal plurality of coextensive helical trenches 130, 132, each trench 130, 132 extending from a respective trench first end 134, 136 to a respective trench second end 138, 140. In the embodiment, the flute first ends 122 and 124 are coextensive with cutting cylinder portion first end 114, and the flute second ends 126, 128 are coextensive with cutting cylinder portion second end 116.

In the embodiment, the bases of flutes 118, 120—corresponding to the depths of trenches 130, 132—are flush with the circumference of guiding tip 106. In the embodiment, flutes 118, 120 and corresponding trenches 130, 132 each extend approximately 360° around the cutting cylinder portion circumference from cutting cylinder first end 114 to cutting cylinder second end 116, providing a relatively shallow flute angle.

The outer edges of flutes 118, 120 define a constant cutting cylinder diameter 160. The cutting cylinder diameter 160 is selected based upon the size of the selected dental implant anchor (or other surgical considerations). In the embodiment, cutting cylinder diameter 160 is 5 mm.

First and second flute second ends 126, 128 are each slightly radiused and beveled at their respective terminal outer edges 142, 144, thereby forming a shoulder transition to guide portion 106.

Guide portion 106 extends from guide portion first end 146 proximate cutting cylinder portion second end 116 to a terminal end 148, defining guide portion length 166. In the embodiment, guide portion length 166 is 3 mm, providing adequate engagement without leaving an excessive requirement of finish drilling. Guide portion 106 includes at least one vent groove 150 extending from a vent groove first end 152 proximate guide portion first end 146 to a vent groove second end 154. Guide portion terminal end 148 approximates a spherically blunted ogival profile. In the embodiment, vent groove is a shallow rounded profile of approximately 0.75 mm depth and 0.75 mm width. Vent groove second end 154 is disposed proximate the base 156 of the ogival profile of terminal end 148 but terminates slightly past the ogive base. In the embodiment, vent groove first end 152 aligns with second trench second end 140 to provide a vent path. By extending from at least one cutting cylinder trench to past the ogival base 156, vent 150 maintains a fluid communication/vent pathway from the region ahead of guided drill bit 100.

Guide portion 106 defines a cylinder of constant diameter 158 from guide portion first end 146 to ogival base 156. The outer surface 164 of guide portion 106 is generally smooth to avoid abrasion and excessive frictional beating, as it is intended to provide alignment for the cutting portion through the pilot hole and not to remove bone material. Guide portion diameter 158 matches the cutting diameter of the selected pilot drill—in the embodiment 2.2 mm.

In the embodiment, depth markings 162 are provided as visual guides for the surgeon.

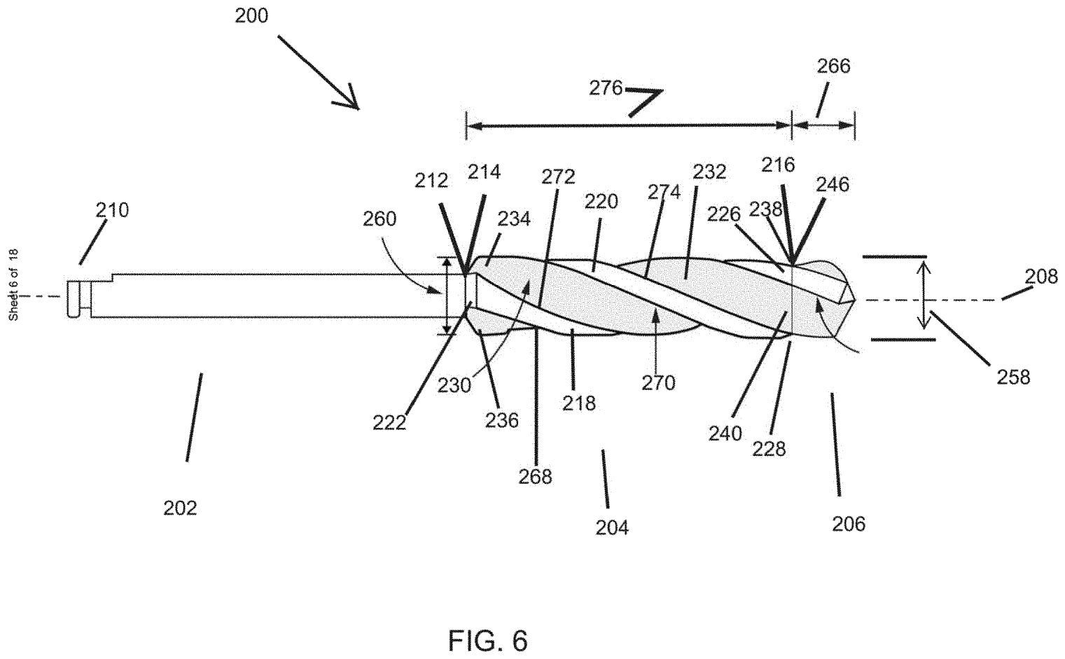

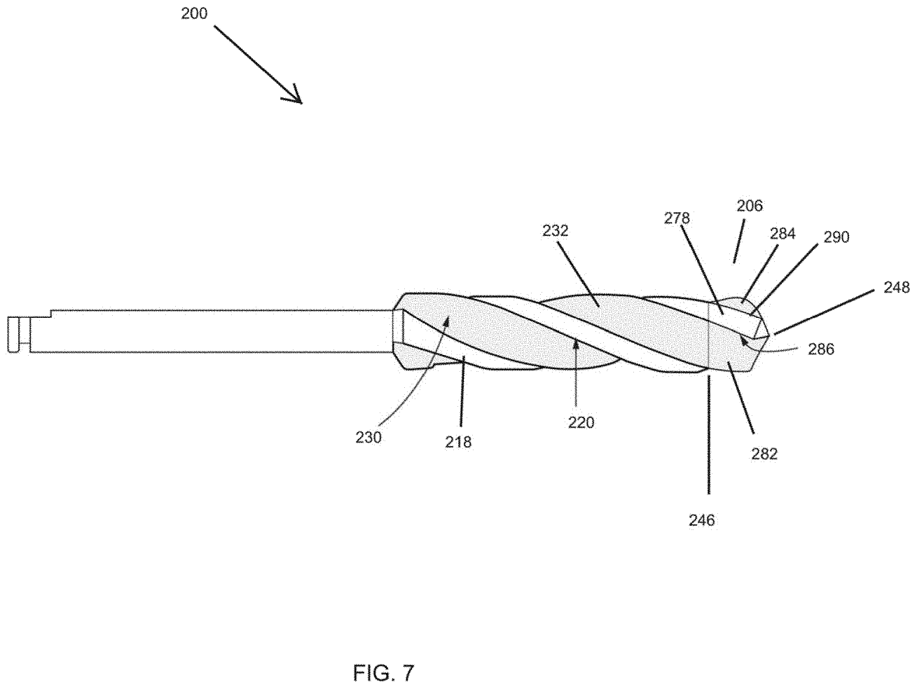

Referring to FIGS. 6-7, an embodiment of a guided implant drill system final drill bit 200 is shown. Final drill bit 200 includes shank portion 202, a guide cylinder portion 204, and a cutting blade tip 206, rigidly connected to each other along rotational axis 208.

Final drill bit shank portion 202 extends from shank portion first end 210 to shank portion second end 212 and is adapted to couple to a selected dental driver or class of dental drivers.

Final drill bit guide cylinder portion 204 extends from guide cylinder portion first end 214, rigidly connected to shank portion second end 212, to guide cylinder portion second end 216, defining a guide cylinder portion length 276. Guide cylinder portion 204 includes a plurality of noncutting helical flutes 218, 220 each extending from a respective guide cylinder flute first end 222, 224 (not visible in view) to a respective guide cylinder flute second end 226, 228. In the embodiment, noncutting guide cylinder flutes 218, 220 are disposed on opposed sides of finishing bit guide cylinder portion 204 at 180° intervals, defining an equal plurality of coextensive guide cylinder portion helical trenches 230, 232, each trench 230, 232 extending from a respective guide cylinder portion trench first end 234, 236 to a respective guide cylinder portion trench second end 238, 240. In the embodiment, the guide cylinder portion flute first ends 222 and 224, and guide cylinder portion trench first ends 234, 236, are coextensive with guide cylinder portion first end 214, and the guide cylinder portion flute second ends 226, 228, and guide cylinder portion trench second ends 238, 240, are coextensive with guide cylinder portion second end 216.

Guided implant final drill bit guide cylinder portion flutes 218, 220 define a constant final drill bit guide cylinder portion diameter 260. Guide cylinder portion flutes 218, 220 are dulled along their respective leading edges 268, 270 and trailing edges 272, 274 (“leading” in the embodiment defined by rotation in the clockwise/right-handed direction).

In the first embodiment, guided implant final drill bit cutting tip 206 extends from a cutting tip first portion 246, which is coextensive with guide cylinder portion second end 216, to a cutting tip terminal end 248, defining a cutting tip depth 266. In the embodiment, cutting tip depth 266 is 3 mm, the same depth as guided implant drill bit guide portion 106. The cutting tip depth 266 is selected to match guided implant drill bit guide portion 106 in order to minimize excess cutting/abrasion in the osteotomy location. Cutting tip diameter 258 is equal to guide cylinder portion diameter 260—each being 5 mm in the embodiment.

In the embodiment, cutting tip 206 includes a plurality of cutting tip flutes 278 and 280 (not visible in view), and corresponding cutting tip trenches 282 and 284, extending from cutting tip first end 246 to cutting tip terminal end 248. Cutting tip flutes 278, 280, each have leading and trailing cutting edges, 286, 288 (not visible in view) and 290, 292 (not visible in view), respectively. In the embodiment, cutting tip flutes 278, 280 are continuous with guide cylinder portion noncutting flutes 218, 220, respectively, and cutting tip trenches 282, 284 are continuous with guide cylinder portion trenches 230, 232, respectively. Trenches 230, 232 in the guide cylinder portion are in fluid communication with cutting tip trenches 282, 284, respectively, allowing the osteotomy location to be irrigated with cooling water during drilling and removing bone particles and tissue from the osteotomy location. The profile of cutting tip terminal end 248 is selected to correspond to the terminal end profile of the selected dental implant anchor.

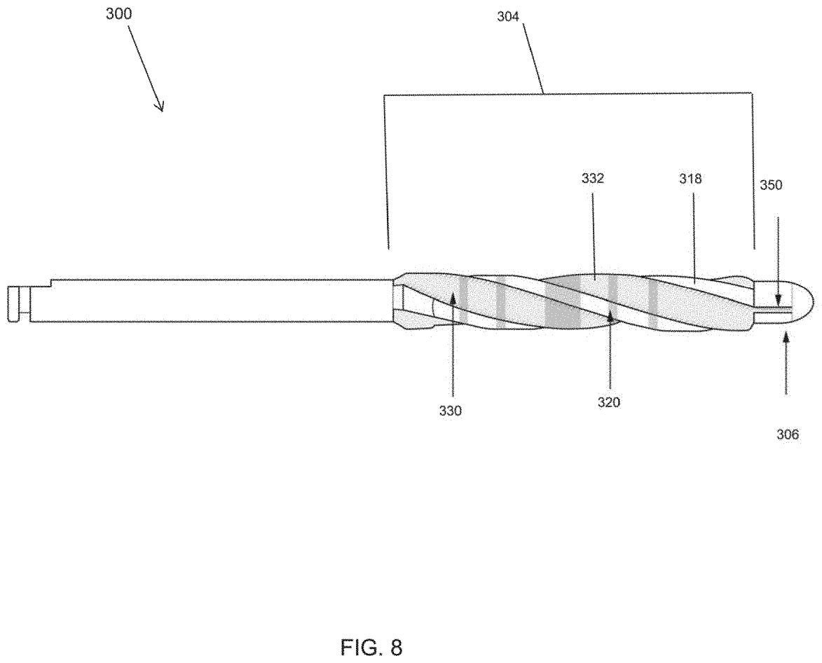

Referring to FIGS. 8-11, additional embodiments of guided implant drill bits 300 and 500, having cutting diameters of 3 mm and 4 mm, respectively, and guided implant final drill bits 400 and 600, having cutting tip diameters of 3 mm and 4 mm, respectively, are shown, demonstrating an embodiment of a guided implant drill bit system. The particular range of drill bits 100, 200 or 300, 400 or 500, 600 are based upon the range of sizes of the dental implant anchor, 5 mm, 3 mm or 4 mm, respectively, which are typical implant anchor sizes. Other diameter sizes within the range 3 mm to 5 mm or may be selected, determined by the dimensions of the dental implant anchor to be implanted. Dental implant anchors outside the range 3 mm to 5 mm are uncommon, but guided implant drill bits and guided implant final drill bits having the same structures for larger or smaller diameters outside this range can be included in the system.

Guided implant drill bit second and third embodiments 300 and 500 include the same structural elements as the first described embodiment 100, but with different cutting diameters. Guided implant drill bit 300 includes a cutting cylinder portion 304 with constant diameter of 3 mm and guide portion 306 having diameter matching the selected pilot drill, in this case 2.2 mm. Guided implant drill bit 300 includes a plurality of helical flutes 318, 320 disposed on opposed sides of cutting cylinder portion 304 at 180° intervals, defining an equal plurality of coextensive helical trenches 330, 332. Guide portion 306 length is 3 mm, providing adequate engagement without leaving an excessive requirement of finish drilling, and includes at least one vent groove 350. Guided implant drill bit 500 includes a cutting cylinder 504 with constant diameter of 4 mm and guide portion 506 having diameter matching the selected pilot drill P, in this case 2.2 mm. Guided implant drill bit 500 includes a plurality of helical flutes 518, 520 disposed on opposed sides of cutting cylinder portion 504 at 180° intervals, defining an equal plurality of coextensive helical trenches 530, 532. Guide portion 506 length is 3 mm, providing adequate engagement without leaving an excessive requirement of finish drilling, and includes at least one vent groove 550.

Guided implant final drill bit second and third embodiments 400 and 600 include the same structural elements as the first described embodiment 200, but with different cutting diameters. Guided implant final drill bit 400 includes noncutting guide cylinder portion 404 with a plurality of noncutting helical flutes 418, 420, defining a constant diameter of 3 mm, and a corresponding plurality of helical trenches 430, 432. Guided implant final drill bit cutting tip 406 defines a cutting tip depth of 3 mm, the same depth as guided implant drill bit guide portion 306. Cutting tip 406 includes a plurality of cutting tip flutes 478 and 480, and corresponding cutting tip trenches 482 and 484. Guided implant final drill bit 600 includes guide cylinder portion 604 with a plurality of noncutting helical flutes 618, 620, defining a constant diameter of 3 mm, and a corresponding plurality of helical trenches 630, 632. Guided implant final drill bit cutting tip 606 defines a cutting tip depth of 3 mm, the same depth as guided implant drill bit guide portion 606. Cutting tip 606 includes a plurality of cutting tip flutes 678 and 680, and corresponding cutting tip trenches 682 and 684.

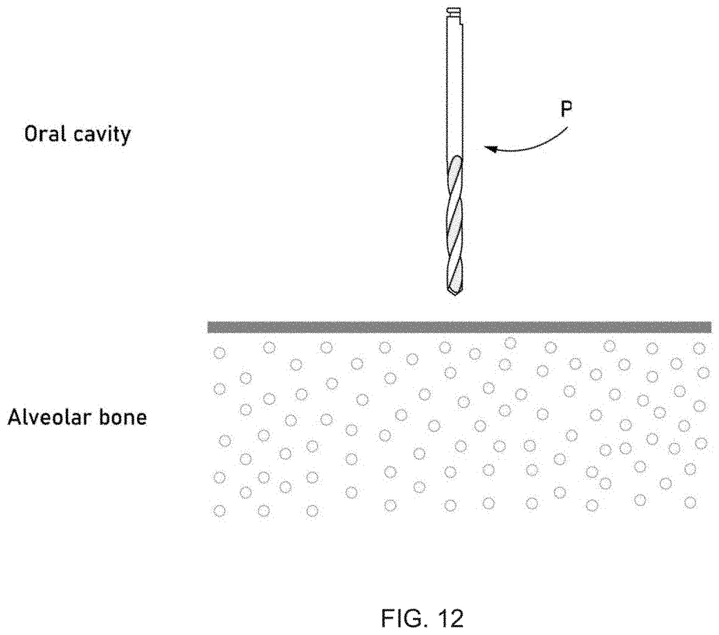

Referring to FIGS. 12-18, a method for performing dental implant anchor preparation surgery is shown, utilizing the 5 mm guided implant drill bit and finishing drill bit, 100 and 200, respectively. The method presumes and does not describe standard surgical practices, such as sterilization, anesthesia, patient preparation and post-drilling steps related to inserting the actual dental implant anchor and implant. Only steps relevant to the method of use of the novel drilling system are described as part of the inventive procedure.

In the procedure (the dental driver is not shown, for simplicity):

a. A pilot hole is created at the implant location using a pilot drill P of selected cutting diameter, in this case 2.2 mm, inserted until a predetermined depth is achieved thereby creating an osteotomy terminal depth T. This may be done with or without resecting the gum tissue, as determined by the surgeon.

b. The surgeon selected a guided implant drill bit 100, 300 or 500, based upon the selected dental implant anchor. In the embodiment of the procedure, the surgeon selects guided implant drill bit 100 for a 5 mm dental implant anchor.

c. Guided implant drill bit 100 is coupled to a selected dental driver (not shown) and used to widen the pilot hole to the desired final diameter. Guided implant drill bit guiding tip 106, having a diameter of 2.2 mm corresponding to the pilot drill bit P, is inserted into the pilot hole, thereby guiding the surgeon during the osteotomy. The surgeon activates the dental driver rotation in the clockwise direction and inserts guided implant drill bit 100 until the terminal end 148 of guided implant drill bit guide portion 106 reaches the osteotomy terminal depth T and is then withdrawn, leaving an osteotomy ridge R which is 3 mm less than the osteotomy terminal depth T.

d. Final drill bit 200, selected to correspond with guided implant drill bit 100 cutting diameter 160 of 5 mm, is coupled to a selected dental driver (not shown). The surgeon activates the dental driver rotation in the clockwise direction and inserts final drill bit 200 into the osteotomy location until cutting tip 206 comes into contact with osteotomy ridge R and continues drilling until final drill bit 200 cutting tip terminal end 248 reaches osteotomy terminal depth T and is then withdrawn.

The osteotomy site would then be ready for the remainder of the implantation procedure for the pre-selected dental implant anchor upon executing the preceding method.

Those skilled in the art will recognize that numerous modifications and changes may be made to the exemplary embodiment(s) without departing from the scope of the claimed invention. It will, of course, be understood that modifications of the invention, in its various aspects, will be apparent to those skilled in the art, some being apparent only after study, others being matters of routine mechanical, chemical and electronic design. No single feature, function or property of the exemplary embodiment(s) is essential. Other embodiments are possible, their specific designs depending upon the particular application. As such, the scope of the invention should not be limited by the particular embodiments herein described but should be defined only by the appended claims and equivalents thereof.

I claim:

1. A first guided implant drill bit having a central axis, the guided implant drill bit further comprising:
a shank portion, a cutting cylinder portion and a guide portion having a guiding tip diameter matching a selected guide drill bit diameter, each aligned along the central axis;
the shank portion adapted to couple to a dental drill driver, the shank extending from a shank first end to a shank second end;
the cutting cylinder portion extending from a cutting cylinder portion first end rigidly connected to the shank portion second end to a cutting cylinder portion second end along the central axis, the cutting cylinder portion including a plurality of flutes defining a plurality of cutting edges, the cutting edges defining the full diameter of the cutting cylinder portion and selected to correspond to a selected dental implant anchor;
the guide portion extending from the cutting cylinder portion second end and including a constant diameter along its axial length and defining a guide portion depth, the guide portion diameter corresponding to a selected pilot drill bit cutting diameter, the guide portion including a smooth noncutting surface, the guide portion further including at least one vent groove extending axially along its surface and a rounded tip and in fluid communication with at least one cutting cylinder trench.

2. A guided implant final drill bit comprising a shank portion connectable to a selected dental driver, a noncutting guide cylinder portion, and a final drill cutting tip, wherein the shank portion, the guide cylinder portion and cutting tip define a central axis, the guided implant final drill further comprising:
the guide cylinder portion extending from a guide cylinder first end rigidly connected to the shank portion second end to a guide cylinder second end connected to the cutting tip and including a plurality of noncutting helical guide cylinder portion flutes and corresponding guide cylinder portion trenches, each respective guide cylinder portion flute and guide cylinder portion trench extending from the guide cylinder portion first end to the guide cylinder portion second end, the guide cylinder portion flutes defining a guide cylinder portion diameter;
the final drill cutting tip extending from a cutting tip base connected to the guide cylinder portion second end to a cutting tip terminal end defining a cutting tip depth, the cutting tip depth corresponding to a selected guided implant drill bit guide portion depth, the cutting tip further including a plurality of cutting tip cutting flutes and corresponding cutting tip trenches extending from the cutting tip base to the cutting tip terminal end, the cutting tip cutting flutes defining a cutting tip diameter which matches the guide cylinder portion diameter, each of the plurality of cutting tip trenches in fluid communication with a corresponding guide cylinder portion trench.

3. A guided implant drill system comprising at least a first set of a guided implant drill bit and a corresponding guided implant final drill bit,
the first set guided implant drill bit further comprising:
a guided implant drill bit shank portion, a cutting cylinder portion and a guide portion, wherein the guided implant drill bit shank portion, cutting cylinder portion and guide portion define a guided implant drill bit central axis, the guided implant drill bit having a guiding tip diameter matching a selected guide drill bit diameter, each aligned along the central axis;
the guided implant drill bit shank portion adapted to couple to a dental drill driver, the guided implant drill bit shank extending from a shank first end to a shank second end;
the cutting cylinder portion extending from a cutting cylinder portion first end rigidly connected to the shank portion second end to a cutting cylinder portion second end along the central axis, the cutting cylinder portion including a plurality of flutes defining a plurality of cutting edges, the cutting edges defining the full diameter of the cutting cylinder portion and selected to correspond to a selected dental implant anchor;
the guide portion extending from the cutting cylinder portion second end and including a constant diameter along its axial length and defining a guide portion depth, the guide portion diameter corresponding to a selected pilot drill bit cutting diameter, the guide portion including a smooth noncutting surface, the guide portion further including at least one vent groove extending axially along its surface and a rounded tip and in fluid communication with at least one cutting cylinder trench.
the first set guided implant final drill bit having a shank portion connectable to a selected dental driver, a noncutting guide cylinder portion, and a final drill cutting tip, wherein the shank portion, the guide cylinder portion and cutting tip define a final drill bit central axis, the first set guided implant final drill bit further comprising:
the guide cylinder portion extending from a guide cylinder first end rigidly connected to the shank portion second end to a guide cylinder second end connected to the cutting tip and including a plurality of noncutting helical guide cylinder portion flutes and corresponding guide cylinder portion trenches, each respective guide cylinder portion flute and guide cylinder portion trench extending from the guide cylinder portion first end to the guide cylinder portion second end, the guide cylinder portion flutes defining a guide cylinder portion diameter which matches the final drill bit cutting tip diameter;
the guided implant final drill bit cutting tip extending from a cutting tip base connected to the guide cylinder portion second end to a cutting tip terminal end defining a cutting tip depth, the cutting tip depth corresponding to the first set guided implant drill bit guide portion depth, the cutting tip further including a plurality of cutting tip cutting flutes and corresponding cutting tip trenches extending from the cutting tip base to the cutting tip terminal end, the cutting tip cutting flutes defining a cutting tip diameter, each of the plurality of cutting tip trenches in fluid communication with a corresponding guide cylinder portion trench.

4. A guided implant drill system as in claim 3, further comprising:
wherein the first set guided implant drill bit cutting cylinder diameter and first set guided implant final drill bit cutting tip diameter are the same and in the range of 3 mm to 5 mm.

5. A guided implant drill system as in claim 3, further comprising:
a plurality of sets of a guided implant drill bit and corresponding guided implant final drill bit, wherein for each guided implant drill bit and corresponding guided implant final drill bit in a respective set the corresponding guided implant drill bit cutting cylinder diameter and guided implant final drill bit cutting tip diameter are the same and selected to match a selected dental implant anchor size.

6. A method for performing dental implant anchor osteotomy, comprising the steps of:
a. providing a guided implant drill system as in claim 3 corresponding to the selected dental implant anchor size;
b. creating a pilot hole at the implant location using the selected pilot drill bit, until a predetermined depth is achieved thereby creating an osteotomy terminal depth at the osteotomy site;
c. using the first set guided implant drill bit coupled to a dental driver, inserting the first set guided implant drill bit guide portion into the pilot hole and drilling into the bone until the guided implant drill bit guide portion terminal end reaches the osteotomy terminal depth, thereby creating an osteotomy ridge; and,
d. using the first set guided implant final drill bit, inserting the first set guided implant final drill bit into the osteotomy site against the osteotomy ridge and drilling until the guided implant final drill bit cutting tip terminal end reaches the osteotomy terminal depth, thereby removing the osteotomy ridge.
